(19) United States
(12) Patent Application Publication
(10) Pub. No.: US 2012/0164681 A1
(43) Pub. Date: Jun. 28, 2012

(54) Method for Detection or Identification of Bacteria or Bacterial Spores

(76) Inventor: Lou Reinisch, Christchurch (NZ)

(21) Appl. No.: 11/850,181

(22) Filed: Sep. 5, 2007

Related U.S. Application Data
(60) Provisional application No. 60/842,245, filed on Sep. 5, 2006.

(51) Int. Cl. G01N 21/64; C12M 1/34

(52) U.S. Cl. 435/34; 435/288.7

(57) ABSTRACT

A method and apparatus for detecting bacteria and bacterial spores are disclosed in which a sample is assessed for the presence of bacteria and/or bacterial spores by reference to horizontally polarized fluorescent light.

### REFERENCE TO PRIOR APPLICATION

This application claims the benefit of U.S. Provisional Application No. 60/842,245, filed Sep. 5, 2006 which is incorporated herein by reference.

### FIELD OF THE INVENTION

The invention relates to an improved fluorescence method for detection and/or identification of bacteria and/or bacterial spores.

### BACKGROUND TO THE INVENTION

Systems for the detection of chemical and biological weaponry are one of increasing international interest. A biological weapon incorporates an organism (bacteria, virus or other disease-causing organism) or toxin found in nature as a weapon of war. Biological warfare agents of critical concern include bacterial spores such as Bacillus anthracis (anthrax), Clostridium tetani (tetanus), and Clostridium Botulinum (botulism). Particularly Bacillus bacteria and Clostridium bacteria form bacterial spores.

Methods for detection of bacteria are known which include exposing a sample which may comprise bacteria to typically UV light, detecting from fluorescence from the sample, and assessing for the presence of light emission at a wavelength or wavelengths distinctive of the bacteria.

Dipicolinic acid (pyridine 2,6 dicarboxylic acid) (DPA) is a major component of bacterial spores and it is unique in that it has only been found in spores. Up to 15% of a spore's dry weight may consist of DPA complexed with calcium ions (CaDPA).

A method for detection of bacterial spores is known which includes combining a lanthanide such as terbium or europium with a sample in which spores may be present, so as to cause the lanthanide to react with the CaDPA naturally present in any spores in the sample, and then determining whether the combined lanthanide—sample medium includes a lanthanide chelate indicative of the presence of bacterial spores by fluorescence testing. In particular the combined lanthanide—sample medium is excited with UV light and is detected for the presence of light emission at a wavelength or wavelengths distinctive of the lanthanide chelate. If the emission intensity at wavelengths distinctive of the lanthanide chelate is above a threshold level, spores are determined to be present in the sample.

U.S. Pat. Nos. 5,701,012 and 5,895,922 disclose a process for detecting the existence of biological particles such as spores whereby fluorescence of the particle is measured and compared against predetermined fluorescence levels.

### OBJECT OF THE INVENTION

It is an object of the present invention to provide an improved or at least alternative fluorescence method for detecting and/or identifying vegetative bacteria and/or bacterial spores.

### SUMMARY OF THE INVENTION

In broad terms in one aspect the invention comprises a method for detecting and/or identifying bacteria and/or bacterial spores by reference to fluorescence, which includes the step of assessing for the presence of, or identifying, bacteria or spores by reference to horizontally polarised fluorescent light.

In broad terms in another aspect the invention comprises a method for in particular detecting bacterial spores by reference to fluorescence which includes the step of assessing for the presence of spores by reference to horizontally polarised fluorescent light.

Preferably the method includes causing the fluorescence to pass a filter oriented to pass substantially only horizontally polarised light.

Preferably the method also includes exciting the fluorescence with vertically polarised ultraviolet light as the excitation light. Alternatively the excitation light may in at least some embodiments of the invention be unpolarised light.

In broad terms in another aspect the invention comprises a detector for detecting and/or identifying bacteria and/or bacterial spores by reference to fluorescence, which is arranged to assess for the presence of, or identify, bacteria or spores by reference to horizontally polarised fluorescent light.

In broad terms in another aspect the invention comprises a detector for in particular detecting bacterial spores by reference to fluorescence, which is arranged to assess for the presence of spores by reference to horizontally polarised fluorescent light.

Preferably the detector includes a filter oriented to pass substantially only horizontally polarised light.

Preferably the detector is arranged to excite the fluorescence with vertically polarised ultraviolet light as the excitation light. Alternatively the excitation light may in at least some embodiments of the invention be unpolarised light.

In one embodiment in broad terms the method includes combining a sample with a lanthanide, exposing the lanthanide—sample combination to light including a wavelength(s) which may excite fluorescence from the lanthanide—sample combination, causing any resulting fluorescence to pass a filter oriented to pass substantially only horizontally polarised light, and assessing for the presence of or identifying spores by reference to the horizontally polarised light.

Typically the lanthanide is terbium. Alternatively, the lanthanide may be europium.

Preferably this method includes exposing the lanthanide—sample combination to vertically polarised light and typically vertically polarised ultraviolet light as the excitation light. Alternatively the excitation light may be unpolarised light.

In one embodiment in broad terms the detector includes a source of excitation light including a wavelength(s) which may excite fluorescence in a combination of a lanthanide and a sample which may comprise spores, a filter oriented to pass substantially only horizontally polarised light in any resulting fluorescence emitted by the sample, and processing means arranged to assess for the presence of or identifying spores, by reference to such horizontally polarised light.

The lanthanide chelate produced such as Tb DPA has fluorescence with a distinctive emission spectrum having sharp emission peaks at the wavelengths referred to previously. We have found that by detecting for the presence of spores and specifically for the presence of a lanthanide chelate resulting from the replacement of Ca in CaDPA by a lanthanide on the combination of a lanthanide and a sample which may contain spores, by exposing the sample to UV light and detecting for fluorescence at one or more wavelengths characteristic of the presence of the lanthanide chelate by reference only to horizontally polarised light emitted, a significant increase in the signal to background or scattered light ratio is obtained.

Optionally a step of determining a threshold emission intensity level above which it is known that the sample medium contains at least some bacteria or spore content can also be carried out.

In another embodiment in broad terms the method includes processing the spores to release DPA from the spores, subjecting the sample to UV radiation, and reassessing the fluorescence of the sample to detect for the presence of or to identify spores, including causing the fluorescence to pass a filter oriented to pass substantially only horizontally polarised light, and assessing and reassessing the fluorescence by reference to the horizontally polarised light.

Preferably this method includes assessing and re-assessing the fluorescence with vertically polarised light and typically vertically polarised ultraviolet light as the excitation light. Alternatively the excitation light may be unpolarised light.

In another embodiment in broad terms the detector includes a UV source, means for processing the spores to release DPA from the spores, means for fluorescence analysis arranged to assess for the presence of or to identify spores by reference to an increase in fluorescence following exposure of the sample to a UV source including a filter oriented to pass substantially only horizontally polarised fluorescent light, and processing means arranged to assess for the presence of or identify spores by reference to such horizontally polarised light.

As used herein the following terms have the meanings given:

“bacteria” (or “vegetative bacteria”) means microscopic, single-celled organisms, including .Bacillus anthracis (anthrax), .Chlostridium tetani (tetanus), and .Chlostridium Botulinum (botulism).

“bacterial spore” means an endospore produced within a bacterium, including spores of .Bacillus anthracis, .Chlostridium tetani, and .Chlostridium Botulinum.

“fluorescence” means the emission of light of a longer wavelength by a source caused by exposure to light of a shorter wavelength from an external source.

“sample” means anything carrying bacteria or spores or in or on which bacteria or spores may reside in whatever form including solid including powder or particulate, on a surface air such as airborne, liquid including in solution or suspension.

“horizontally polarised light” means light partially or primarily polarised perpendicular to a scattering plane.

“vertically polarised” means light polarised parallel to a scattering plane.

“identification” includes partial identification such as identification of a genus or class if not species of bacteria or bacterial spores, so that identification also includes classification of bacteria or bacterial spores.

The term “comprising” as used in this specification means “consisting at least in part of”. When interpreting statements in this specification which include that term, the features, prefaced by that term in each statement, all need to be present but other features can also be present. Related terms such as “comprise” and “comprised” are to be interpreted in the same manner.

### BRIEF DESCRIPTION OF THE FIGURES

In the following description figures are referred to as follows:

FIG. 1: is a plot of intensity against emission wavelength of fluorescence which is referred to in the subsequent description of experimental work.

FIG. 2: is a plot of intensity against emission wavelength as in FIG. 1 but with peaks removed from the background.

FIG. 3: is a plot of intensity against emission wavelength for isolated peaks.

### DETAILED DESCRIPTION OF THE INVENTION

The method of the invention for detecting and/or identifying bacteria and/or bacterial spores by reference to fluorescence, includes the step of assessing for the presence of bacteria or spores by reference to horizontally polarised fluorescent light. We have found that the resolution obtained when only the horizontally polarised fluorescent light is detected is significantly superior. That is, a significantly superior signal to background light ratio is obtained. This in turn enhances the discrimination that can be achieved with fluorescence detection or identification methods.

Since in methods for detection and/or identification of bacteria and/or bacterial spores using fluorescence, the background light comprises mostly Rayleigh and Mie scattering from the sample, that the step of assessing or identifying by reference to horizontally polarised fluorescent light, to enhance the signal to background ratio, can be used with any such fluorescence method, with or without chemical reagents, to detect and/or identify bacteria and/or bacterial spores.

In one method of the invention a sample which is suspected of containing bacterial spores is combined with a lanthanide. The lanthanide is preferably prepared in solution form, and is then combined with the sample medium. Typically the sample medium is then heated. If bacterial spores are present in the sample medium, the lanthanide reacts with the CaDPA in the spores to produce a lanthanide chelate, specifically terbium dipicolinate where the lanthanide is Tb. The combined lanthanide—sample medium is excited with energy within the excitation spectra, preferably at the absorbance peaks of the lanthanide chelate. The excitation energy may be supplied for example by a UV laser or lamp. For terbium dipicolinate the preferred excitation energy is in the ranges of 270±5 nm and/or 278±5 nm. Emission wavelengths for terbium dipicolinate include emission peaks at ranges of 490±10 nm, 546±10 nm, 586±10 nm, and 622±10 nm. The method includes detecting one or more of these wavelengths. We have found that the resolution obtained when only the horizontally polarised fluorescent light is detected is significantly superior. That is, a significantly superior signal to background light ratio is obtained.

In another method of the invention DPA/CaDPA in spores is released by breakdown of the spore structure, by heating in water for example, to release the DPA/CaDPA into the supernatant liquid (“the sample”). This method then comprises assessing the fluorescence of the sample, exposing the sample to ultraviolet radiation, preferably of wavelength in the range 250-350 nm most preferably about 280 nm, and re-assessing the fluorescence of the sample, and determining the presence (or absence) of spores. If the fluorescence is increased after exposure to UV radiation the sample is assessed as containing bacterial spores. In the assessment and reassessment of fluorescence the sample is preferably exposed to UV in the wavelength range 300-400 nm and fluorescence is detected in the wavelength range 300-500 nm.

UV light sources include lamps (including fluorescent lamps, gas lamps, tungsten filament lamps, quartz lamps, halogen lamps, arc lamps, and pulsed discharge lamps, for example), and UV light emitting diodes, laser diodes, laser of any type capable of producing UV radiation (such as gas, dye or solid state). Light sources may or may not need filtering, or could simply be filtered by gratings, interference fitters or coloured glass filters. They could also be filtered by cut-off filters. Two-photon techniques may be employed where two separate photons of differing wavelength are used to provide the required excitation wavelength. For example, a 280 nm light necessary to bring about fluorescence enhancement can be achieved from a high intensity of 560 nm light. Two photons of 560 nm could be simultaneously absorbed to create the same effect and response as one 280 nm photon being absorbed. An advantage of such a two-photon absorption is that all optics and the light emitter work in the visible region of the spectrum, whilst the absorption band of the sample is in the UV region. It should be appreciated that when we refer to subjecting the sample to UV radiation, scenarios such as this are included. It is the absorption band which should be considered in this case.

A detection system may include means for analysis of the fluorescence. Such means may include computer processing apparatus which, for example records and compares or analyses actual fluorescence measurements or may determine the difference between a first and subsequent recording, to determine if any fluorescence enhancement has been observed. The analysis means may record and store the outputs or it may simply trigger an alarm for example, if bacteria or spores (greater than a threshold limit) are detected.

In methods of the invention, if the irradiating light is polarised, although the scattered (background) radiation will preserve the polarisation of this light, the fluorescence signal does not. Thus if the sample is irradiated with vertically polarised light, although the scattered light will remain vertically polarised, that light which is fluoresced from the sample (as a result of the presence of the bacteria or spores) is a mixture of horizontally polarised and vertically polarised radiation. By measurement of only the horizontally polarised light, fluorescence is measured with little, if any, background scattering. Alternatively the excitation light may be unpolarised light, and only the horizontally polarised fluorescent light is measured. Suitable apparatus can be arranged by incorporation of polarising filters into known apparatus. At least a horizontally polarising filter before the detector, and a vertically polarising filter may also be employed with the UV source.

The terms “vertical” and “horizontal” polarisation are used with reference to the “scattering surface”. In preferred forms we use the 90 degree geometry between incident and scattered light, and the incident radiation is polarised vertically (with respect to the scattering surface or phase). The scattering surface or phase will depend upon the nature of the sample being investigated but is the molecule or species responsible for reflecting and/or absorbing the incident light.

In one embodiment of the invention a simple detector may be used to observe only fluorescence and thus indicate whether or not bacteria or spores are present. In an alternative embodiment, using a more specialised detector which resolves the intensity of emission as a function of wavelength, the shape of the fluorescence can be analysed to determine what or species of bacterial spores are present in a sample.

Some embodiments of the invention may take advantage of the phenomenon that fluorescence has a distinct lifetime. This lifetime is relative to that of the scattered light, which has a zero lifetime. Specifically, after light is absorbed by the spore it takes a short amount of time for the fluorescence to occur. This is usually between 0.1-10 ns. Thus in general terms if following a short pulsed excitation, emitted light having a zero lifetime is ignored and other emitted light detected, the contribution to the emission by scattering is reduced and thus the signal to noise ratio improved.

An alternative means of taking advantage of this phenomenon involves modulating the intensity of the light, for example in a sinusoidal fashion. The fluorescence exhibited by the bacteria or spores may follow the modulation of the exciting light, delayed by the fluorescence lifetime. Thus in this embodiment a modulated fluorescence signal is detected (again for example a sine wave type signal, if the exciting light was modulated accordingly) delayed by the fluorescence lifetime.

The sensitivity of the detector can be set to ignore a few bacteria or spores that occur naturally. Biological weaponry such as anthrax requires approximately 10,000 anthrax spores to lethally infect a person with a 50% probability. Thus the detection limit may be set at for example 100 spores. This is well above the background level for spores, and 1000 times lower than the level needed to lethally infect individuals. Although many bacterial spores are relative harmless to humans, others cause gastrointestinal problems and others (like anthrax) are deadly. The levels of bacterial spores should almost always be quite low in the environment thus the detection of bacterial spores above a given threshold level would more than likely signal bioterrorism.

The invention has importance in the bioterrorism field however there are many other applications as would be known to one skilled in the art. Examples include (but are not limited to) the situation in New Zealand where MAF has sprayed certain areas with Bacillus bacterial spores as an insecticide against unwanted pests. The method of the invention and a detector of the invention could be employed to detect levels of exposure which would be severely detrimental to the public or such susceptible persons, or to show which regions are safe for such susceptible persons to occupy during spraying.

A further important application of the method and detector of the invention is identifying and quantifying bacterial spores in dried products such as foodstuffs. One particular application is identification and quantification of Bacillus bacterial spores in milk powder. Milk powder providers, even with their best precautions, may still have contamination by bacterial spores in their product. Regulatory authorities set guidelines as to what is a minimum spore level for safe use and consumption by the public. Different thresholds will be appropriate for different end uses of the powder. Thus a convenient method of determining whether or not there is a spore presence and what level of presence would be advantageous. The method of the invention is suitable for such an application.

The method of the invention may also be used for detecting spores in a water supply or an air supply, in various medical applications, and in fuels, for example.

The method helps to separate the bacterial spore fluorescence from the fluorescence of other materials for example those found in dust. Thus this enhances discrimination.

### EXAMPLE

The following example of experimental work illustrates the invention in relation to detection of bacterial spores:

Terbium oxide was dissolved in excess in water. Bacillus globigii spores were then added to the water and the sample was heated in an autoclave and cooled. The fluorescence was measured with a horizontal polariser on the emission.

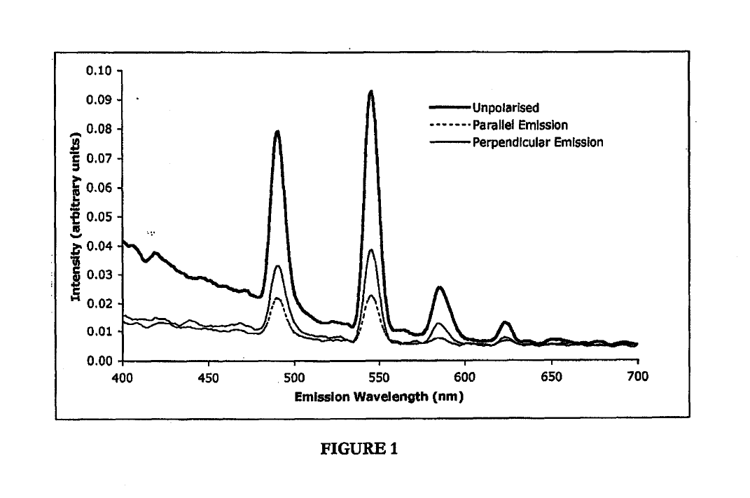

FIG. 1 shows fluorescence without and with polariser. The fluorometer used was set for 3 sec per point and 1 nm per point for the emission scan. The PMT high voltage was 1000 v.

Terbium-DPA is excited at 280 nm and the 4 peaks are at about 490 nm, 540 nm, 590 nm and 620 nm. It is clear from FIG. 1 that the signal to noise or the signal to background is maximal for the horizontal polarisation of the emission light.

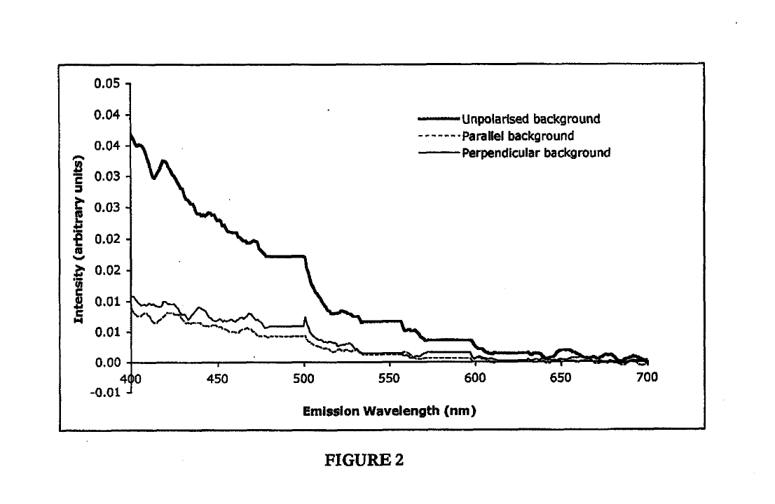

To numerically justify this, the peaks were removed from the background and the constant value was subtracted from the backgrounds. This is illustrated in FIG. 2.

The integrated area under the curves shown in FIG. 2 is:

Unpolarised background: 3.54 a.u.-nm

Vertically polarised background: 0.81 a.u.-nm

Horizontally polarised background: 1.08 a.u.-nm

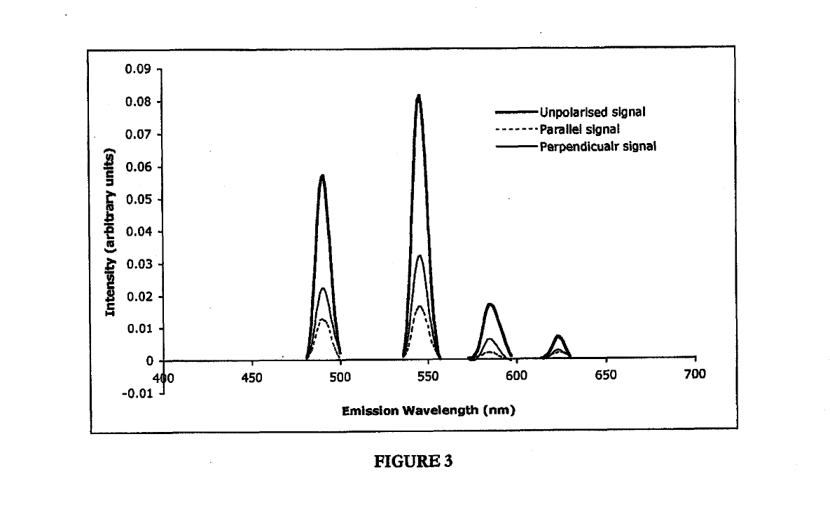

FIG. 3 shows the actual peaks having been isolated. The integrated area under the curves is:

Unpolarised peaks: 1.61 a.u.-nm

Vertically polarised peaks: 0.34 a.u.-nm

Horizontally polarised peaks: 0.63 a.u.-nm

The ratio of the integrated peak intensity to the background intensity is:

Unpolarised ratio: 0.45

Vertically polarised ratio: 0.41

Horizontally polarised ratio: 0.58

In summary by detecting only the horizontal polarisation of the fluorescence light an increase of approximately 30% in the signal to background ratio is obtained.

Alternatively considering the area under the peaks versus the area under the background curve at only those wavelengths were we are measuring the peaks gives for the ratio of the integrated peak intensity to the background intensity:

Unpolarised ratio: 2.69

Vertically polarised ratio: 2.81

Horizontally polarised ratio: 3.36

This is a 25% improvement in the signal to background ratio.

The foregoing describes the invention by way of example and with reference to particular embodiments, it is to be understood that modifications and/or improvements may be made without departing from the scope or spirit of the invention as defined in the accompanying claims.

1. A method for detecting bacterial spores in a sample by reference to fluorescence which includes the steps of exposing the sample to light to excite fluorescence from the sample and assessing the sample for the presence of spores by reference to the intensity of fluorescent light if any at one or more wavelengths, which further includes assessing the sample for the presence of spores by reference to horizontally polarised fluorescent light to increase discrimination between fluorescent light excited from the sample and reflected light.

2-3. (canceled)

4. A method according to claim 1 which includes exposing the sample to vertically polarised light as the excitation light.

5. A method according to claim 1 which includes assessing the fluorescence at a wavelength or wavelengths in one or more of the wavelength ranges of 490±10 nm, 546±10 nm, 586±10 nm, and 622±10 nm.

6. A method according to claim 1 wherein the spores comprise spores of any one or more of Bacillus anthracis, Chlostridium tetanci, and Chlostridium Botulinum.

7. A detector for detecting bacterial spores in a sample by reference to fluorescence, which is arranged to expose the sample to light to excite fluorescence from the sample and assess for the presence of spores by reference to the intensity of horizontally polarised fluorescent light to increase discrimination between fluorescent light excited from the sample and reflected light.

8-9. (canceled)

10. A detector according to claim 7 arranged to expose the sample to vertically polarised light as the excitation light.

11. A detector according to claim 7 arranged to assess the fluorescence at a wavelength or wavelengths in one or more of the wavelength ranges of 490±10 nm, 546±10 nm, 586±10 nm, and 622±10 nm.

12. A method for detecting bacteria in a sample by reference to fluorescence which includes the steps of exposing the sample to light to excite fluorescence from the sample and assessing for the presence of bacteria by reference to the intensity of fluorescent light if any at one or more wavelengths, which further includes assessing the sample for the presence of spores by reference to horizontally polarised fluorescent light to increase discrimination between fluorescent light excited from the sample and reflected light.

13. (canceled)

14. A method according to claim 12 which includes exposing the sample to vertically polarised light as the excitation light.

15. A method according to claim 12 which includes assessing the fluorescence at a wavelength or wavelengths in one or more of the wavelength ranges of 490±10 nm, 546±10 nm, 586±10 nm, and 622±10 nm.

16. A method according to claim 12 wherein the bacteria comprise any one or more of Bacillus anthracis, Chlostridium tetanci, and Chlostridium Botulinum.

17-31. (canceled)
